(19) United States
(12) Patent Application Publication
(10) Pub. No.: US 2026/0298901 A1
(43) Pub. Date: Oct. 1, 2026

(54) SYSTEMS AND METHODS FOR DETECTION AND QUANTIFICATION OF POLLEN ON PLANT SURFACES

(71) Applicant: AgZen Inc., Somerville, MA (US)

(72) Inventor: Vishnu Jayaprakash, Somerville, MA (US)

(21) Appl. No.: 19/631,291

(22) Filed: Mar. 27, 2026

Related U.S. Application Data
(60) Provisional application No. 63/779,819, filed on Mar. 28, 2025.

(51) Int. Cl. G01N 33/00; G06V 20/10; G06V 20/17

(52) U.S. Cl. CPC G01N 33/0098; G06V 20/17; G06V 20/188; G06V 20/194

(57) ABSTRACT

Presented herein are systems and methods for automatically detecting and/or quantifying the amount of pollen on plant surfaces (e.g., determining how much pollen has been deposited onto plants, e.g., a region of plants). In certain embodiments, the plants are seed corn plants, where pollen grains are each from about 80 micrometers to about 125 micrometers in diameter, e.g., from about 90 micrometers to about 100 micrometers in diameter. It has been found that the methods described herein are able to accurately determine the amount of such plant surface that is covered by seed corn pollen. For example, in certain embodiments, it can be determined whether a density of coverage has been achieved to facilitate successful pollination. This information may be used, for example, to predict yields, determine which varieties in the crop were (or were likely) successfully pollinated, where and/or when artificial pollination methods are recommended, and the like.

### CROSS REFERENCE TO RELATED APPLICATIONS

This application claims priority to and the benefit of U.S. Provisional Patent Application No. 63/779,819, filed Mar. 28, 2025, the disclosure of which is incorporated by reference herein in its entirety.

### FIELD

This invention relates generally to agricultural systems and methods. More particularly, in certain embodiments, the invention relates to systems and methods for detecting and/or quantifying pollen on plant surfaces (e.g., determining how much pollen has been deposited onto plants, e.g., a region of plants, e.g., a crop of plants, e.g., the silks and/or leaves of the plants in a crop, e.g., the female part of plants in a crop).

### BACKGROUND

A corn plant is a cereal grass, with the produced corn being considered a seed or grain, that is, the harvested dry seed or fruit of a cereal grass. Seed corn refers to a type of corn that is specifically grown and harvested for use as seeds for planting. Seed corn production may involve cross-pollinating different corn varieties to create a hybrid with improved traits such as yield, disease resistance, and pest tolerance.

Corn breeding is facilitated by wind and occurs when pollen from the male part (e.g., tassel) of a plant is carried to the female part (e.g., silks, ears) of the same or nearby plants. Corn plants have both male and female reproductive structures. While corn can self-pollinate, it is more common for corn plants to be pollinated by pollen from neighboring plants (e.g., cross-pollination). Corn pollen is relatively large. For example, a grain of pollen can have a diameter from about 80 micrometers to about 125 micrometers, e.g., from about 90 micrometers to about 100 micrometers. This is large compared to other grasses, whose diameter is from about 20 to 25 micrometers; thus, corn pollen doesn’t travel as far as pollen from other grasses, but can still be carried by the wind for some distance. Each tassel produces millions of grains of pollen, and pollination success varies depending on factors such as timing, weather (e.g., wind, temperature, humidity, drought, and the like), and pollen density.

Pollination success is important to achieving a successful yield of corn (or other grain). Farmers may employ various methods to achieve successful pollination, for example, planting corn in various crop configurations (e.g., in blocks), and/or by performing artificial pollination (e.g., on-demand crop pollination). Artificial pollination may be conducted using pollen-collecting machines that drive through fields of actively pollen-shedding plants where pollen is pulled off tassels in male rows, treated and/or preserved in a lab, then applied to plants at an optimal time (e.g., by dropping pollen from a helicopter, a drone, or by depositing the pollen in some other way), thereby increasing yield. An example of an artificial pollination method is described in U.S. Patent No. 10,905,060, entitled “Seed Production,” the text of which is incorporated herein by reference in its entirety.

Pollination success, whether natural or artificial, is often highly dependent on the amount of pollen that actually collects onto the surface of the corn plant (or other grain plant). This may impact, for example, the determination of whether, when, and/or how to proceed with either natural or artificial pollination, and it may inform recommendations for future crops.

Currently, pollen counting is more often performed for assessment of allergy risks rather than for agricultural applications. Pollen counting is generally performed via air sampling techniques. Examples of air sampling methods include the use of “rotorod samples” which are greased silicone rods that collect pollen in the air over a specific period of time, and use of a “Berkard trap” which draws in air at a specific rate and collects pollen on an adherent film.

Such air sampling methods for determining a pollen count are generally unsatisfactory for determining whether pollen has actually made it onto the female part of a plant. As a result, farmers generally perform manual random sampling of corn silks to determine the extent to which pollen has made it onto the plant and assess likelihood of successful pollination of a certain region of plants (e.g., a certain crop, or one or more portions of a crop). In some situations, the random sampling informs whether, when, and/or where harvested pollen should be deposited onto plants to achieve successful pollination. These sampling techniques are often time consuming and inaccurate, given that many samples may be necessary to obtain an accurate assessment over a large region of plants.

Accordingly, there is a need for reliable and more accurate technologies for detecting and quantifying pollen on plants.

### SUMMARY

Presented herein are systems and methods for automatically detecting and/or quantifying pollen on plant surfaces (e.g., determining how much pollen has been deposited onto plants, e.g., a region of plants, e.g., a crop of plants, e.g., the silks and/or leaves of the plants in a crop, e.g., the female part of plants in a crop).

In one aspect, the invention is directed to a system for automatically detecting and/or quantifying pollen coverage on target plant surfaces in a given region of interest (e.g., determining a density, coverage percentage, or other quantity measurement of pollen that has been deposited onto plants, e.g., onto a region of plants, e.g., a crop of plants, e.g., the silks and/or leaves of the plants in a crop, e.g., the female part of plants in a crop, e.g., silks of seed corn plants), the system comprising: one or more sensors [(e.g., optical or non-optical) (e.g., of a type such as a visible wavelength imaging sensor such as a camera such as a red-green-blue (RGB) camera, charged-coupled display (CCD) camera, or a complementary metal-oxide-semiconductor (CMOS) camera, an infrared wavelength imaging sensor such as a Light Detection and Ranging (LiDAR) sensor or a shortwave infrared (SWIR) camera, and/or a radio detection and ranging (Radar) sensor)], wherein the one or more sensors captures (e.g., in real time) data (e.g., image data) reflecting a state of the plant surfaces in the given region of interest (e.g., wherein the sensor or group of sensors are mounted to or otherwise physically attached to or within a tractor, spreader, or other agricultural vehicle); a processor of a computing device; and a memory having instructions stored thereon, wherein the instructions, when executed by the processor, cause the processor to: receive the data (e.g., image data) captured by the one or more sensors reflecting the state of the plant surfaces; and compute a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest, e.g., the uniformity of pollen distribution over the swath of the target plant surfaces in the given region of interest.

In certain embodiments, the one or more sensors comprises at least one overhead sensor positioned on or in (a) a tower or other structure, and/or (b) one or more drones (unmanned aerial vehicle), and/or (c) a manned aerial vehicle (e.g., a manned helicopter), wherein the at least one sensor captures overhead images (e.g., visible spectrum, infrared, radio waves, or otherwise) of the target plant surfaces in the given region of interest.

In certain embodiments, the one or more sensors comprises at least one overhead sensor and at least one camera and/or other sensor mounted to a tractor, spreader, or other agricultural vehicle.

In certain embodiments, the one or more sensors comprises one or more flying drones (e.g., remote controlled drones, global positioning system (GPS) drones, fixed wind drones, multi-rotor drones, single-rotor helicopter drones, and/or fixed-wing hybrid VTOL (vertical take-off and landing) drones).

In certain embodiments, the one or more sensors is capable of capturing images of a region of interest that spans at least 1 m², at least 10 m², at least 50 m², at least 100 m², at least 1000 m², at least 0.01 km², at least 0.1 km², or at least 1 km².

In certain embodiments, the plant surfaces in the given region of interest comprise, or consist of, or consist essentially of, the female part of plants in a crop (e.g., silks of seed corn plants) and wherein the pollen grains have an average diameter from about 80 micrometers to about 125 micrometers, e.g., from about 90 micrometers to about 100 micrometers, wherein the one or more sensors comprises a visible wavelength imaging sensor and/or an infrared wavelength imaging sensor.

In certain embodiments, the one or more sensors comprises a red-green-blue (RGB) camera.

In certain embodiments, the one or more sensors comprises a short-wave infrared (SWIR) camera.

In certain embodiments, at least one of the one or more sensors is mounted to or otherwise physically attached to or within a tractor, spreader, or other agricultural vehicle.

In certain embodiments, the crop comprises silks of seed corn plants.

In certain embodiments, the processor automatically determines the value of pollen coverage using a trained machine learning module.

In another aspect, the invention is directed to a method for automatically detecting and/or quantifying pollen coverage on target plant surfaces in a given region of interest (e.g., determining a density, coverage percentage, or other quantity measurement of pollen that has been deposited onto plants, e.g., onto a region of plants, e.g., a crop of plants, e.g., the silks and/or leaves of the plants in a crop, e.g., the female part of plants in a crop, e.g., silks of seed corn plants), the method comprising: capturing, by one or more sensors [(e.g., optical or non-optical) (e.g., of a type such as a visible wavelength imaging sensor such as a camera such as a red-green-blue (RGB), charged-coupled display (CCD) camera, or a complementary metal-oxide-semiconductor (CMOS) camera, an infrared wavelength imaging sensor such as a Light Detection and Ranging (LiDAR) sensor or a shortwave infrared (SWIR) camera, and/or a radio detection and ranging (Radar) sensor)] (e.g., in real time) data (e.g., image data) reflecting a state of the plant surfaces in the given region of interest (e.g., wherein the sensor or group of sensors are mounted to or otherwise physically attached to or within a tractor, spreader, or other agricultural vehicle); automatically receiving, by a processor of a computing device, the data (e.g., the image data) captured by the one or more sensors reflecting the state of the plant surfaces; and automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest, e.g., the uniformity of pollen distribution over the swath of the target plant surfaces in the given region of interest.

In certain embodiments, the one or more sensors comprises at least one overhead sensor positioned on or in (a) a tower or other structure, and/or (b) one or more drones (unmanned aerial vehicle), and/or (c) a manned aerial vehicle (e.g., a manned helicopter), wherein the at least one sensor captures overhead images (e.g., visible spectrum, infrared, radio waves, or otherwise) of the target plant surfaces in the given region of interest.

In certain embodiments, the one or more sensors comprises at least one overhead sensor and at least one camera and/or other sensor mounted to a tractor, spreader, or other agricultural vehicle.

In certain embodiments, the one or more sensors comprises one or more flying drones (e.g., remote controlled drones, global positioning system (GPS) drones, fixed wind drones, multi-rotor drones, single-rotor helicopter drones, and/or fixed-wing hybrid VTOL (vertical take-off and landing) drones).

In certain embodiments, the one or more sensors captures images of a region of interest that spans at least 1 m², at least 10 m², at least 50 m², at least 100 m², at least 1000 m², at least 0.01 km², at least 0.1 km², or at least 1 km².

In certain embodiments, the plant surfaces in the given region of interest comprise, or consist of, or consist essentially of, the female part of plants in a crop (e.g., silks of seed corn plants) and wherein the pollen grains have an average diameter from about 80 micrometers to about 125 micrometers, e.g., from about 90 micrometers to about 100 micrometers.

In certain embodiments, the one or more sensors comprises a visible wavelength imaging sensor and/or an infrared wavelength imaging sensor.

In certain embodiments, the one or more sensors comprises a red-green-blue (RGB) camera.

In certain embodiments, the one or more sensors comprises an infrared wavelength imaging sensor.

In certain embodiments, the one or more sensors comprises a Short-Wavelength InfraRed (SWIR) camera.

In certain embodiments, at least one of the one or more sensors is mounted to or otherwise physically attached to or within a tractor, spreader, or other agricultural vehicle.

In certain embodiments, the processor automatically determines the value of pollen coverage using a trained machine learning module.

In another aspect, the invention is directed to a system for automatically detecting and/or quantifying pollen coverage on target plant surfaces in a given region of interest, the system comprising: a processor; and a memory having instructions thereon, wherein the instructions, when executed by the processor, cause the processor to receive data captured by one or more sensors reflecting a state of plant surfaces in the given region of interest; and automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

In certain embodiments, the processor automatically determines the value of pollen coverage using a trained machine learning module.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1 illustrates a method for automatically detecting and/or quantifying pollen coverage on target plant surfaces in a given region of interest, according to an illustrative embodiment.

FIG. 2 is a block diagram of an exemplary system described herein, according to an illustrative embodiment.

FIG. 3 is a schematic showing an implementation of a network environment for use in providing systems, methods, and architectures as described herein, according to an illustrative embodiment.

FIG. 4 is a schematic showing exemplary computing devices that can be used to implement the techniques described, according to an illustrative embodiment.

### DESCRIPTION OF THE INVENTION

It is contemplated that systems, architectures, devices, methods, and processes of the present claims encompass variations and adaptations developed using information from embodiments described herein. Adaptation and/or modification of the systems, architectures, devices, methods, and processes described herein may be performed, as contemplated by this description.

Throughout the description, where articles, devices, and systems are described as having, including, or comprising specific components, or where processes and methods are described as having, including, or comprising specific steps, it is contemplated that, additionally, there are articles, devices, systems, and architectures of the present disclosure that consist essentially of, or consist of, the recited components, and that there are processes and methods according to the present invention that consist essentially of, or consist of, the recited processing steps.

It should be understood that the order of steps or order for performing certain actions is immaterial so long as the invention remains operable. Moreover, two or more steps or actions may be conducted simultaneously.

The mention herein of any publication is not an admission that the publication serves as prior art with respect to any of the claims presented herein. The Background section may include concepts informed by the embodiments recited in the claims and further described elsewhere in the specification. The discussion of concepts in the Background section is not an admission that the subject matter discussed is prior art.

Headers are provided for the convenience of the reader – the presence and/or placement of a header is not intended to limit the scope of the subject matter described herein.

Presented herein are systems and methods for automatically detecting and/or quantifying pollen on plant surfaces (e.g., determining how much pollen has been deposited onto plants, e.g., a region of plants, e.g., a crop of plants, e.g., the silks and/or leaves of the plants in a crop, e.g., the female part of plants in a crop). In certain embodiments, the plants are seed corn plants, where pollen grains are each from about 80 micrometers to about 125 micrometers in diameter, e.g., from about 90 micrometers to about 100 micrometers in diameter.

It has been found that the methods and systems described herein are able to accurately determine the amount of plant surface that is covered by pollen. For example, in certain embodiments, it can be determined whether a density of coverage (e.g., an average number of pollen grains per silk) has been achieved to facilitate successful pollination. This information may be used, for example, to predict yields, determine which varieties in the crop were (or were likely) successfully pollinated, where and/or when artificial pollination methods are recommended, and the like.

In certain embodiments, systems and methods presented herein automatically detect and/or quantify pollen coverage on target plant surfaces (e.g., determining a density, coverage percentage, or other quantity measurement of pollen that has been deposited onto plants, e.g., onto a region of plants, e.g., a crop of plants, e.g., the silks and/or leaves of the plants in a crop, e.g., the female part of plants in a crop, e.g., silks of seed corn plants). In certain embodiments, the measurement system includes (i) a sensor or group of sensors (e.g., optical or non-optical) (e.g., of a type such as a visible wavelength imaging sensor such as a camera (e.g., a red-green-blue (RGB) camera, charged-coupled display (CCD) camera, a complementary metal-oxide-semiconductor (CMOS) camera), an infrared wavelength imaging sensor such as a Light Detection and Ranging (LiDAR) sensor or a shortwave infrared (SWIR) camera, and/or a radio detection and ranging (Radar) sensor), that capture (e.g., in real time) the state of the plant surface (e.g., wherein the sensor or group of sensors are mounted to or otherwise physically attached to or within a tractor, spreader, or other agricultural vehicle); and (ii) an algorithm or group of algorithms and a processor for processing data captured by the sensor or group of sensors to compute the quantity of pollen that has been deposited onto the plant surfaces (e.g., compute pollen coverage in terms of (a) an absolute or relative covered surface area, (b) a number of pollen grains or total pollen volume for a given region of interest, and/or (c) a measure of the uniformity of pollen distribution for the region of interest, e.g., the uniformity of pollen distribution over the swath of the target surface).

In certain embodiments, the sensor or group of sensors include at least one sensor positioned on or in (a) a tower or other structure, and/or (b) one or more drones (unmanned aerial vehicle), and/or (c) a manned aerial vehicle (e.g., a manned helicopter), wherein the at least one sensor captures overhead images (e.g., visible spectrum, infrared, radio waves, or otherwise) of the target surface (e.g., including the region(s) of interest). In certain embodiments, the one or more drones are flying drones (e.g., remote controlled drones, global positioning system (GPS) drones, fixed wind drones, multi-rotor drones, single-rotor helicopter drones, and/or fixed-wing hybrid VTOL (vertical take-off and landing) drones). In certain embodiments, the one or more sensors is capable of capturing images of a region of interest that spans at least 1 m², at least 10 m², at least 50 m², at least 100 m², at least 1000 m², at least 0.01 km², at least 0.1 km², or at least 1 km².

In other embodiments, one or more of the overhead sensor systems described above is/are coupled with one or more cameras and/or other sensors mounted to a tractor, spreader, or other agricultural vehicle.

In certain embodiments, data captured by one or more sensors reflecting the state of a target agricultural surface can include data captured by environmental sensors. Environmental sensors can be used to capture environmental data corresponding to one or more environmental conditions at a location and at a time images are obtained. Exemplary environmental sensors include temperature sensors, humidity sensors, pressure sensors, wind sensors, light sensors, air quality sensors, gas sensors, rainfall sensors, radiation sensors, and soil sensors. It is also possible to account for environmental conditions such as wind, temperature, humidity, plant density, plant density variability, and the like, using such sensors.

### Exemplary Method

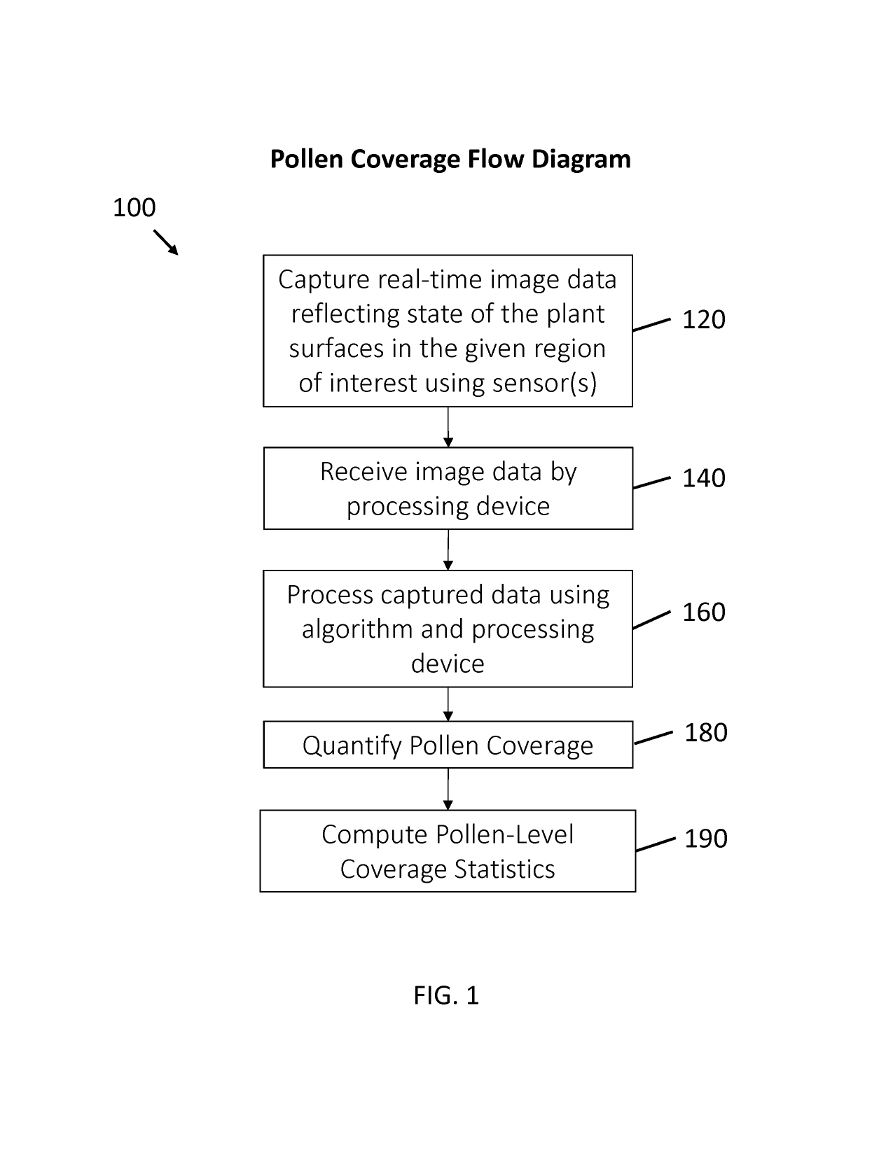

FIG. 1 illustrates a method 100 for automatically detecting and/or quantifying pollen coverage on target plant surfaces in a given region of interest, according to the present disclosure. At step 120, the method 100 includes capturing real-time image data reflecting the state of the plant surfaces in the given region of interest using sensor(s). At step 140, the method 100 includes receiving image date by the processing device(s). At step 160, the method 100 includes processing the captured data using one or more algorithms and the processing device(s). At step 180, the method 100 includes quantifying the pollen coverage. At step 190, the method 100 includes computing one or more pollen-level coverage statistics.

### Exemplary System

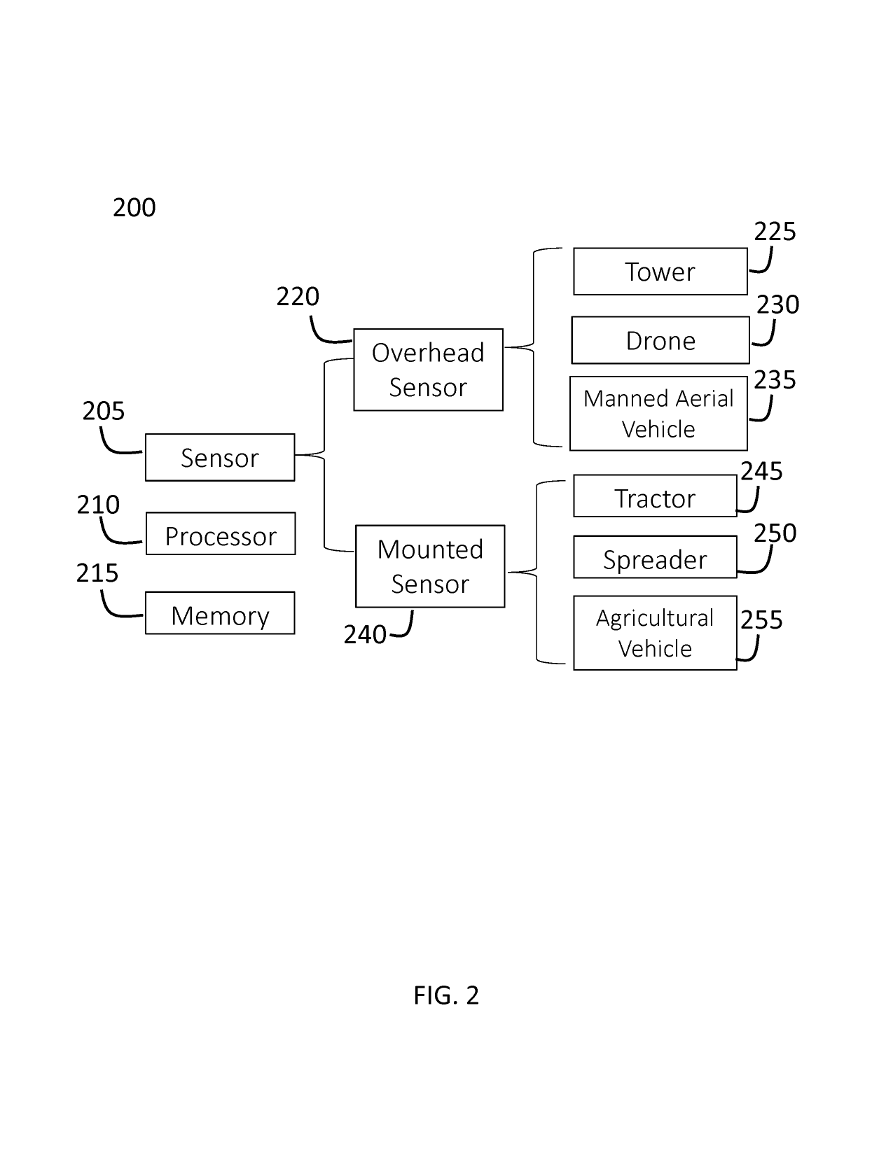

FIG. 2 illustrates a system 200 for automatically detecting and/or quantifying pollen coverage on target plant surfaces in a given region of interest, according to the present disclosure.

The system 200 includes one or more sensors 205 such as a visible wavelength imaging sensor such as a camera (e.g., a red-green-blue (RGB) camera, charged-coupled display (CCD) camera, or a complementary metal-oxide-semiconductor (CMOS) camera), an infrared wavelength imaging sensor such as a Light Detection and Ranging (LiDAR) sensor or a shortwave infrared (SWIR) camera, or a radio detection and ranging (Radar) sensor. Sensors are used to capture (e.g., in real time) data (e.g., image data) reflecting a state of the plant surfaces in the given region of interest.

The system 200 includes a processor of a computing device 210 and a memory 215 with stored instructions.

In certain embodiments, the system 200 includes one or more overhead sensors 220 positioned on or in (a) a tower 225 or other structure, (b) a drone (e.g., unmanned aerial vehicles) 230, or (c) a manned aerial vehicle 235. For the avoidance of doubt, the system 200 can include one or more (a) towers or other structures, (b) drones, and (c) manned aerial vehicles. Overhead sensors are used to capture overhead images of target plant surfaces in a given region of interest.

In certain embodiments, the system 200 includes at least one overhead sensor 220 and at least one mounted sensor 240. In certain embodiments, a mounted sensor is one which has been mounted to or otherwise physically attached to a tractor 245, a spreader 250, or other agricultural vehicle 255.

### Software, Computer System, and Network Environment

Certain embodiments described herein make use of computer algorithms in the form of software instructions executed by a computer processor. In certain embodiments, the software instructions include a machine learning module, also referred to herein as artificial intelligence (AI) software. As used herein, a machine learning module refers to a computer implemented process (e.g., a software function) that implements one or more specific machine learning techniques, e.g., artificial neural networks (ANNs), e.g., convolutional neural networks (CNNs), random forest, decision trees, support vector machines, and the like, in order to determine, for a given input, one or more output values. In certain embodiments, the input comprises alphanumeric data which can include numbers, words, phrases, or lengthier strings, for example. In certain embodiments, the one or more output values comprise values representing numeric values, words, phrases, or other alphanumeric strings.

In certain embodiments, machine learning modules implementing machine learning techniques are trained, for example using datasets that include categories of data described herein. Such training may be used to determine various parameters of machine learning algorithms implemented by a machine learning module, such as weights associated with layers in neural networks. In certain embodiments, once a machine learning module is trained, e.g., to accomplish a specific task such as identifying certain response strings, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In certain embodiments, machine learning modules may receive feedback, e.g., based on user review of accuracy, and such feedback may be used as additional training data, to dynamically update the machine learning module. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of an ANN module may be carried out via specialized hardware (e.g., via an application specific integrated circuit (ASIC)).

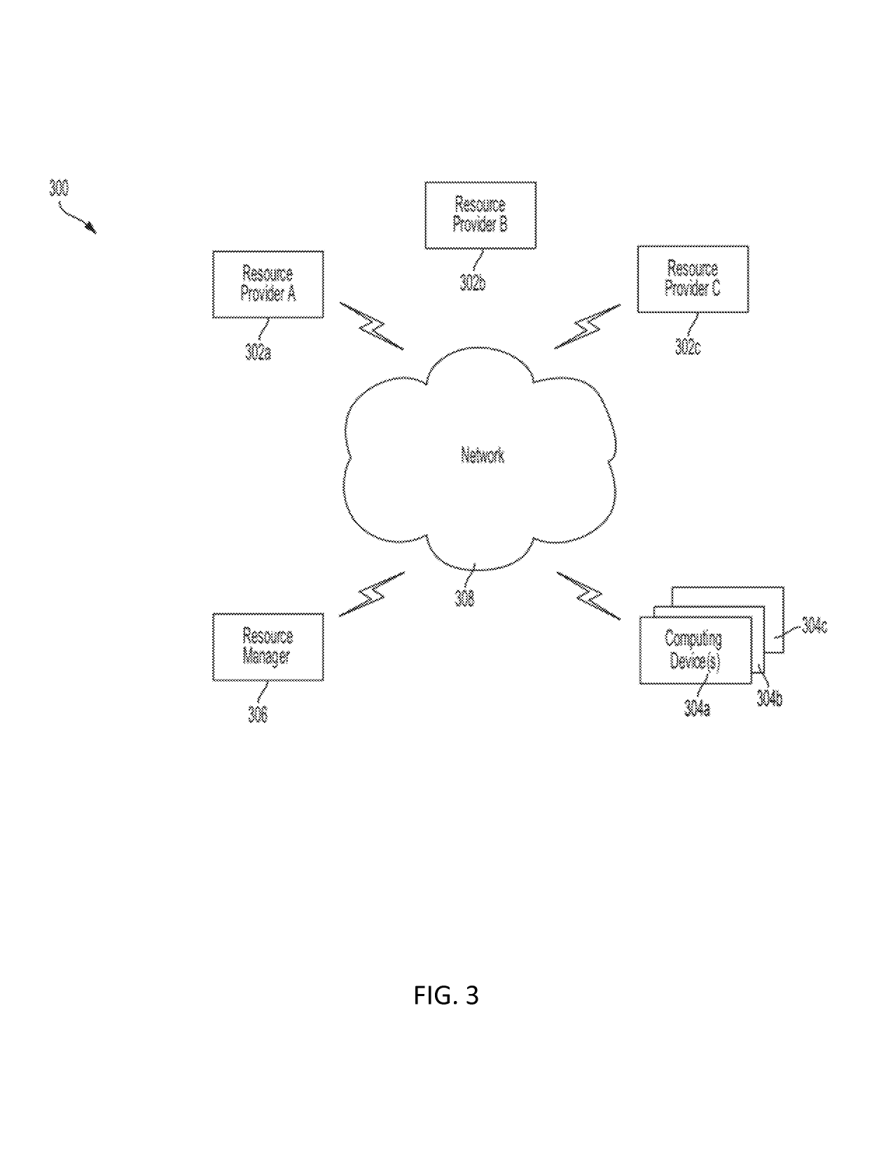

As shown in FIG. 3, an implementation of a network environment 300 for use in providing systems, methods, and architectures as described herein is shown and described. In brief overview, referring now to FIG. 3, a block diagram of an exemplary cloud computing environment 300 is shown and described. The cloud computing environment 300 may include one or more resource providers 302a, 302b, 302c (collectively, 302). Each resource provider 302 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 302 may be connected to any other resource provider 302 in the cloud computing environment 300. In some implementations, the resource providers 302 may be connected over a computer network 308. Each resource provider 302 may be connected to one or more computing device 304a, 304b, 304c (collectively, 304), over the computer network 308.

The cloud computing environment 300 may include a resource manager 306. The resource manager 306 may be connected to the resource providers 302 and the computing devices 304 over the computer network 308. In some implementations, the resource manager 306 may facilitate the provision of computing resources by one or more resource providers 302 to one or more computing devices 304. The resource manager 306 may receive a request for a computing resource from a particular computing device 304. The resource manager 306 may identify one or more resource providers 302 capable of providing the computing resource requested by the computing device 304. The resource manager 306 may select a resource provider 302 to provide the computing resource. The resource manager 306 may facilitate a connection between the resource provider 302 and a particular computing device 304. In some implementations, the resource manager 306 may establish a connection between a particular resource provider 302 and a particular computing device 304. In some implementations, the resource manager 306 may redirect a particular computing device 304 to a particular resource provider 302 with the requested computing resource.

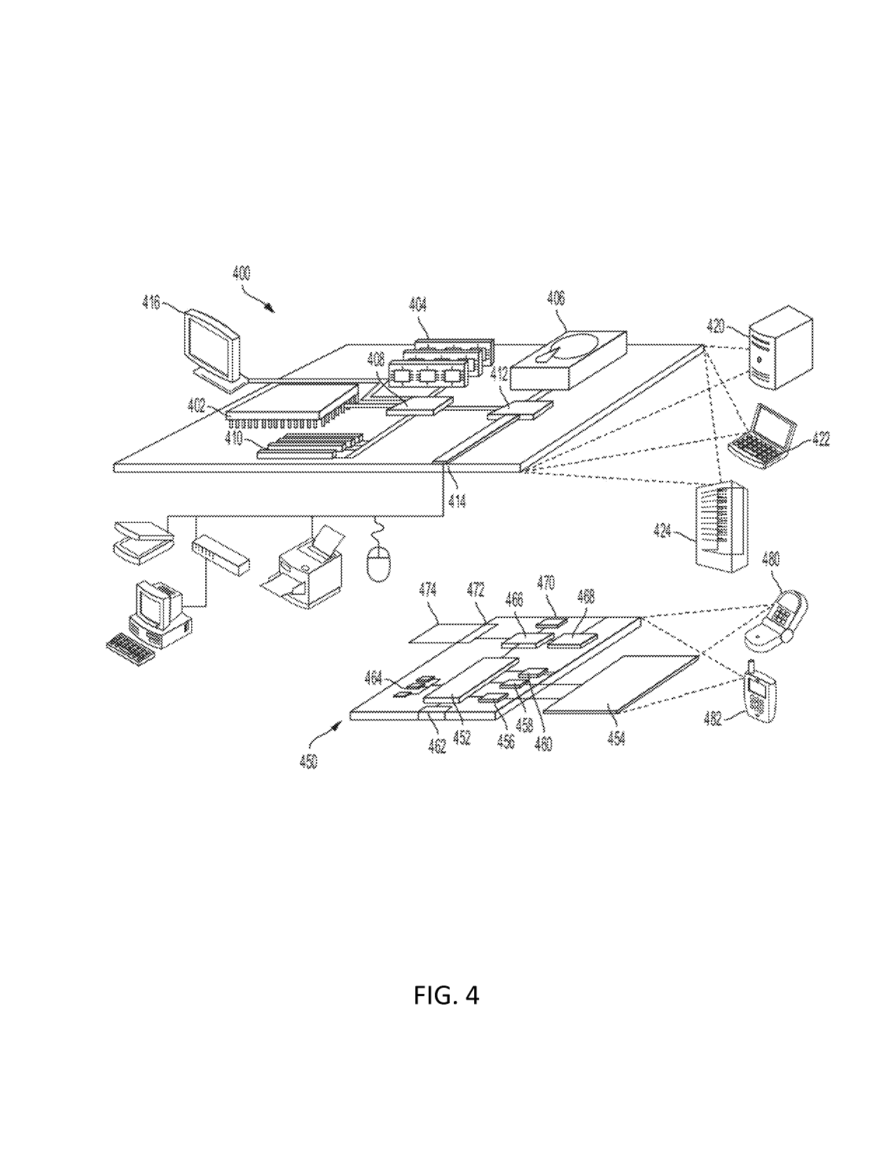

FIG. 4 shows an example of a computing device 400 and a mobile computing device 450 that can be used to implement the techniques described in this disclosure. The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 404 stores information within the computing device 400. In some implementations, the memory 404 is a volatile memory unit or units. In some implementations, the memory 404 is a non-volatile memory unit or units. The memory 404 may also be another form of computer-readable medium, such as a magnetic or optical disk.

The storage device 406 is capable of providing mass storage for the computing device 400. In some implementations, the storage device 406 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 402), perform one or more methods, such as those described above. The instructions can also be stored by one or more storage devices such as computer- or machine-readable mediums (for example, the memory 404, the storage device 406, or memory on the processor 402).

The high-speed interface 408 manages bandwidth-intensive operations for the computing device 400, while the low-speed interface 412 manages lower bandwidth-intensive operations. Such allocation of functions is an example only. In some implementations, the high-speed interface 408 is coupled to the memory 404, the display 416 (e.g., through a graphics processor or accelerator), and to the high-speed expansion ports 410, which may accept various expansion cards (not shown). In the implementation, the low-speed interface 412 is coupled to the storage device 406 and the low-speed expansion port 414. The low-speed expansion port 414, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 400 may be implemented in a number of different forms, as shown in the figure. For example, it may be implemented as a standard server 420, or multiple times in a group of such servers. In addition, it may be implemented in a personal computer such as a laptop computer 422. It may also be implemented as part of a rack server system 424. Alternatively, components from the computing device 400 may be combined with other components in a mobile device (not shown), such as a mobile computing device 450. Each of such devices may contain one or more of the computing device 400 and the mobile computing device 450, and an entire system may be made up of multiple computing devices communicating with each other.

The mobile computing device 450 includes a processor 452, a memory 464, an input/output device such as a display 454, a communication interface 466, and a transceiver 468, among other components. The mobile computing device 450 may also be provided with a storage device, such as a micro-drive or other device, to provide additional storage. Each of the processor 452, the memory 464, the display 454, the communication interface 466, and the transceiver 468, are interconnected using various buses, and several of the components may be mounted on a common motherboard or in other manners as appropriate.

The processor 452 can execute instructions within the mobile computing device 450, including instructions stored in the memory 464. The processor 452 may be implemented as a chipset of chips that include separate and multiple analog and digital processors. The processor 452 may provide, for example, for coordination of the other components of the mobile computing device 450, such as control of user interfaces, applications run by the mobile computing device 450, and wireless communication by the mobile computing device 450.

The processor 452 may communicate with a user through a control interface 458 and a display interface 456 coupled to the display 454. The display 454 may be, for example, a TFT (Thin-Film-Transistor Liquid Crystal Display) display or an OLED (Organic Light Emitting Diode) display, or other appropriate display technology. The display interface 456 may comprise appropriate circuitry for driving the display 454 to present graphical and other information to a user. The control interface 458 may receive commands from a user and convert them for submission to the processor 452. In addition, an external interface 462 may provide communication with the processor 452, so as to enable near area communication of the mobile computing device 450 with other devices. The external interface 462 may provide, for example, for wired communication in some implementations, or for wireless communication in other implementations, and multiple interfaces may also be used.

The memory 464 stores information within the mobile computing device 450. The memory 464 can be implemented as one or more of a computer-readable medium or media, a volatile memory unit or units, or a non-volatile memory unit or units. An expansion memory 474 may also be provided and connected to the mobile computing device 450 through an expansion interface 472, which may include, for example, a SIMM (Single In Line Memory Module) card interface. The expansion memory 474 may provide extra storage space for the mobile computing device 450, or may also store applications or other information for the mobile computing device 450. Specifically, the expansion memory 474 may include instructions to carry out or supplement the processes described above, and may include secure information also. Thus, for example, the expansion memory 474 may be provide as a security module for the mobile computing device 450, and may be programmed with instructions that permit secure use of the mobile computing device 450. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 452), perform one or more methods, such as those described above. The instructions can also be stored by one or more storage devices, such as one or more computer- or machine-readable mediums (for example, the memory 464, the expansion memory 474, or memory on the processor 452). In some implementations, the instructions can be received in a propagated signal, for example, over the transceiver 468 or the external interface 462.

The mobile computing device 450 may communicate wirelessly through the communication interface 466, which may include digital signal processing circuitry where necessary. The communication interface 466 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 468 using a radio-frequency. In addition, short-range communication may occur, such as using a Bluetooth®, Wi-Fi™, or other such transceiver (not shown). In addition, a GPS (Global Positioning System) receiver module 470 may provide additional navigation- and location-related wireless data to the mobile computing device 450, which may be used as appropriate by applications running on the mobile computing device 450.

The mobile computing device 450 may also communicate audibly using an audio codec 460, which may receive spoken information from a user and convert it to usable digital information. The audio codec 460 may likewise generate audible sound for a user, such as through a speaker, e.g., in a handset of the mobile computing device 450. Such sound may include sound from voice telephone calls, may include recorded sound (e.g., voice messages, music files, etc.) and may also include sound generated by applications operating on the mobile computing device 450.

The mobile computing device 450 may be implemented in a number of different forms, as shown in the figure. For example, it may be implemented as a cellular telephone 480. It may also be implemented as part of a smart-phone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system and/or device(s) can include clients and servers. A client and server are generally remote from each other and typically interact through a communication network. The relationship of client and server arises by virtue of computer programs running on the respective computers and having a client-server relationship to each other.

In some implementations, certain modules described herein can be separated, combined or incorporated into single or combined modules. Any modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

While the invention has been particularly shown and described with reference to specific preferred embodiments, it should be understood by those skilled in the art that various changes in form and detail may be made therein without departing from the spirit and scope of the invention as defined by the appended claims.

### EMBODIMENTS

1. A system for automatically detecting and/or quantifying pollen coverage on target plant surfaces in a given region of interest, the system comprising:

one or more sensors, wherein the one or more sensors captures data reflecting a state of the plant surfaces in the given region of interest;

a processor of a computing device; and

a memory having instructions stored thereon, wherein the instructions, when executed by the processor, cause the processor to:

receive the data captured by the one or more sensors reflecting the state of the plant surfaces; and

compute a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

2. The system of embodiment 1, wherein the one or more sensors comprises at least one overhead sensor positioned on or in (a) a tower or other structure, and/or (b) one or more drones, and/or (c) a manned aerial vehicle, wherein the at least one sensor captures overhead images of the target plant surfaces in the given region of interest.

3. The system of embodiment 1 or 2, wherein the one or more sensors comprises at least one overhead sensor and at least one camera and/or other sensor mounted to a tractor, spreader, or other agricultural vehicle.

4. The system of any one of the previous embodiments, wherein the one or more sensors comprises one or more flying drones.

5. The system of any one of the previous embodiments, wherein the one or more sensors is capable of capturing images of a region of interest that spans at least 1 m², at least 10 m², at least 50 m², at least 100 m², at least 1000 m², at least 0.01 km², at least 0.1 km², or at least 1 km².

6. The system of any one of the previous embodiments, wherein the plant surfaces in the given region of interest comprise, or consist of, or consist essentially of, the female part of plants in a crop and wherein the pollen grains have an average diameter from about 80 micrometers to about 125 micrometers.

7. The system of any one of the previous embodiments, wherein the one or more sensors comprises a visible wavelength imaging sensor and/or an infrared wavelength imaging sensor.

8. The system of any one of the previous embodiments, wherein the one or more sensors comprises a red-green-blue (RGB) camera.

9. The system of any one of the previous embodiments, wherein the one or more sensors comprises an infrared wavelength imaging sensor.

10. The system of any one of the previous embodiments, wherein the one or more sensors comprises a Short-Wavelength InfraRed (SWIR) camera.

11. The system of any one of the previous embodiments, wherein at least one of the one or more sensors is mounted to or otherwise physically attached to or within a tractor, spreader, or other agricultural vehicle.

12. The system of any one of the previous embodiments, wherein the crop comprises silks of seed corn plants.

13. The system of any one of the previous embodiments, wherein the processor automatically determines the value of pollen coverage using a trained machine learning module.

14. A method for automatically detecting and/or quantifying pollen coverage on target plant surfaces in a given region of interest, the method comprising:

capturing, by one or more sensors, data reflecting a state of the plant surfaces in the given region of interest;

automatically receiving, by a processor of a computing device, the data captured by the one or more sensors reflecting the state of the plant surfaces; and

automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

15. The method of embodiment 14, wherein the one or more sensors comprises at least one overhead sensor positioned on or in (a) a tower or other structure, and/or (b) one or more drones, and/or (c) a manned aerial vehicle, wherein the at least one sensor captures overhead images of the target plant surfaces in the given region of interest.

16. The method of embodiment 14 or 15, wherein the one or more sensors comprises at least one overhead sensor and at least one camera and/or other sensor mounted to a tractor, spreader, or other agricultural vehicle.

17. The method of any one of embodiments 14-16, wherein the one or more sensors comprises one or more flying drones.

18. The method of any one of embodiments 14-17, wherein the one or more sensors capture images of a region of interest that spans at least 1 m², at least 10 m², at least 50 m², at least 100 m², at least 1000 m², at least 0.01 km², at least 0.1 km², or at least 1 km².

19. The method of any one of embodiments 14-18, wherein the plant surfaces in the given region of interest comprise, or consist of, or consist essentially of, the female part of plants in a crop and wherein the pollen grains have an average diameter from about 80 micrometers to about 125 micrometers.

20. The method of any one of embodiments 14-19, wherein the one or more sensors comprises a visible wavelength imaging sensor and/or an infrared wavelength imaging sensor.

21. The method of any one of embodiments 14-20, wherein the one or more sensors comprises a red-green-blue (RGB) camera.

22. The method of any one of embodiments 14-21, wherein the one or more sensors comprises an infrared wavelength imaging sensor.

23. The method of any one of embodiments 14-22, wherein the one or more sensors comprises a Short-Wavelength InfraRed (SWIR) camera.

24. The method of any one of embodiments 14-23, wherein at least one of the one or more sensors is mounted to or otherwise physically attached to or within a tractor, spreader, or other agricultural vehicle.

25. The method of any one of embodiments 14-24, wherein the processor automatically determines the value of pollen coverage using a trained machine learning module.

26. A system for automatically detecting and/or quantifying pollen coverage on target plant surfaces in a given region of interest, the system comprising:

a processor; and

a memory having instructions thereon, wherein the instructions, when executed by the processor, cause the processor to

receive data captured by one or more sensors reflecting a state of plant surfaces in the given region of interest; and

automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

27. The system of embodiment 26, wherein the processor automatically determines the value of pollen coverage using a trained machine learning module.

What is claimed is:

1. A system for automatically detecting and/or quantifying pollen coverage on target plant surfaces in a given region of interest, the system comprising:
one or more sensors, wherein the one or more sensors captures data reflecting a state of the plant surfaces in the given region of interest;
a processor of a computing device; and
a memory having instructions stored thereon, wherein the instructions, when executed by the processor, cause the processor to:
receive the data captured by the one or more sensors reflecting the state of the plant surfaces; and
compute a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

2. The system of claim 1, wherein the one or more sensors comprises at least one overhead sensor positioned on or in (a) a tower or other structure, and/or (b) one or more drones, and/or (c) a manned aerial vehicle, wherein the at least one sensor captures overhead images of the target plant surfaces in the given region of interest.

3. The system of claim 1, wherein the one or more sensors comprises at least one overhead sensor and at least one camera and/or other sensor mounted to a tractor, spreader, or other agricultural vehicle.

4. The system of claim 1, wherein the one or more sensors comprises one or more flying drones.

5. The system of claim 1, wherein the one or more sensors are capable of capturing images of a region of interest that spans at least 1 m² at least 10 m² at least 50 m² at least 100 m² at least 1000 m² at least 0.01 km² at least 0.1 km² or at least 1 km².

6. The system of claim 1, wherein the plant surfaces in the given region of interest comprise, or consist of, or consist essentially of, the female part of plants in a crop and wherein the pollen grains have an average diameter from about 80 micrometers to about 125 micrometers.

7. The system of claim 6, wherein the crop comprises silks of seed corn plants.

8. The system of claim 1, wherein the one or more sensors comprises a visible wavelength imaging sensor and/or an infrared wavelength imaging sensor.

9. The system of claim 1, wherein the one or more sensors comprises a red-green-blue (RGB) camera.

10. The system of claim 1, wherein the one or more sensors comprises an infrared wavelength imaging sensor.

11. The system of claim 1, wherein the one or more sensors comprises a Short-Wavelength InfraRed (SWIR) camera.

12. The system of claim 1, wherein at least one of the one or more sensors is mounted to or otherwise physically attached to or within a tractor, spreader, or other agricultural vehicle.

13. The system of claim 1, wherein the processor automatically determines the value of pollen coverage using a trained machine learning module.

14. A method for automatically detecting and/or quantifying pollen coverage on target plant surfaces in a given region of interest, the method comprising:
capturing, by one or more sensors, data reflecting a state of the plant surfaces in the given region of interest;
automatically receiving, by a processor of a computing device, the data captured by the one or more sensors reflecting the state of the plant surfaces; and
automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

15. The method of claim 14, wherein the one or more sensors comprises at least one overhead sensor positioned on or in (a) a tower or other structure, and/or (b) one or more drones, and/or (c) a manned aerial vehicle, wherein the at least one sensor captures overhead images of the target plant surfaces in the given region of interest.

16. The method of claim 14, wherein the one or more sensors comprises at least one overhead sensor and at least one camera and/or other sensor mounted to a tractor, spreader, or other agricultural vehicle.

17. The method of claim 14, wherein the one or more sensors comprises one or more flying drones.

18. The method of claim 14, wherein the one or more sensors captures images of a region of interest that spans at least 1 m² at least 10 m² at least 50 m² at least 100 m² at least 1000 m² at least 0.01 km² at least 0.1 km² or at least 1 km².

19. The method of claim 14, wherein the plant surfaces in the given region of interest comprise, or consist of, or consist essentially of, the female part of plants in a crop and wherein the pollen grains have an average diameter from about 80 micrometers to about 125 micrometers.

20. The method of claim 14, wherein the one or more sensors comprises a visible wavelength imaging sensor and/or an infrared wavelength imaging sensor.

21. The method of claim 14, wherein the one or more sensors comprises a red-green-blue (RGB) camera.

22. The method of claim 14, wherein the one or more sensors comprises an infrared wavelength imaging sensor.

23. The method of claim 14, wherein the one or more sensors comprises a Short-Wavelength InfraRed (SWIR) camera.

24. The method of claim 14, wherein at least one of the one or more sensors is mounted to or otherwise physically attached to or within a tractor, spreader, or other agricultural vehicle.

25. The method of claim 14, wherein the processor automatically determines the value of pollen coverage using a trained machine learning module.

26. A system for automatically detecting and/or quantifying pollen coverage on target plant surfaces in a given region of interest, the system comprising:
a processor; and
a memory having instructions thereon, wherein the instructions, when executed by the processor, cause the processor to
receive data captured by one or more sensors reflecting a state of plant surfaces in the given region of interest; and
automatically determining, by the processor, a value of pollen coverage in terms of any one or more of (a)-(c) as follows: (a) an absolute or relative covered surface area over the given region of interest, (b) an average number of pollen grains or total pollen volume for the given region of interest, and/or (c) a measure of the uniformity of pollen distribution for the given region of interest.

27. The system of claim 26, wherein the processor automatically determines the value of pollen coverage using a trained machine learning module.
